(19) United States
(12) Patent Application Publication
(10) Pub. No.: US 2015/0330980 A1
(43) Pub. Date: Nov. 19, 2015

(54) METHODS AND KITS FOR THE DETECTION OF VIRAL INFECTIONS

(71) Applicant: SMART Biotech Ltd., Rehovot (IL)

(72) Inventor: Tamar JEHUDA-COHEN, Moshav Gimzo (IL)

(21) Appl. No.: 14/383,572

(22) PCT Filed: Mar. 7, 2013
(86) PCT No.: PCT/IL2013/050210

§ 371 Date: Sep. 8, 2014

Related U.S. Application Data
(60) Provisional application No. 61/612,419, filed on Mar. 19, 2012.
(60) Provisional application No. 61/608,120, filed on Mar. 8, 2012.

(51) Int. Cl. G01N 33/569; C07K 16/10; C07K 16/08

(52) U.S. Cl. CPC G01N 33/56983; C07K 16/08; C07K 16/1045; C07K 16/1036; C07K 16/109; C07K 16/082; G01N 2469/10; G01N 2333/16; G01N 2333/15; G01N 2333/186; G01N 2333/02

(57) ABSTRACT

The invention relates to an improved assay for detecting antibodies in a tissue sample from individuals who test seronegative by conventional assay techniques, thus aiding in the diagnosis of possible pathogenic infections. Specifically, the invention relates to improved assay methods and kits that enable efficient detection of antibodies against a viral infection.

### FIELD OF THE INVENTION

The invention relates to an improved assay for detecting antibodies in a tissue sample from individuals who test seronegative by conventional assay techniques, thus aiding in the diagnosis of possible pathogenic infections. Specifically, the invention relates to improved assay methods and kits that enable efficient detection of antibodies against a viral infection.

### BACKGROUND OF THE INVENTION

Serological detection of antibodies against a variety of infectious disease agents is considered evidence of exposure to and/or active infection by the agent. There are different assays for detecting specific antibodies such as lateral-flow assays (Rapid assays), enzyme-linked immunosorbent assay (e.g. ELISA, EIA) and immunoblots. Some commercial kits are commonly used as screening tests for serological detection of antibodies, others are used for diagnosis, with several different algorithms for confirming an initially reactive result. Other methods, using detection methods other than the appearance of color can also be used, such as immunofluorescence, chemiluminescence, luminescence, carbon nanotubes, changes in the current or the light beam properties due to the antibody binding, and many more may also be applicable. Polymerase chain reaction (PCR) technique may also be used for detection of infection.

Serological screening techniques are being utilized worldwide for the detection of human immunodeficiency virus type (HIV), simian immunodeficiency virus (SIV), xenotropic murine leukemia virus (XMRV), Human T-cell lymphotrophic virus (HTLV-1), hepatitis A, B, C, D, and E viruses, Herpes simplex virus, cytomegalovirus, Epstein-Barr virus, tuberculosis, etc.

The presence of antibody against a virus is considered a strong indicator of an infection. Immunoassays, such as ELISA and Rapid assays, are currently being utilized on blood samples (including serum, plasma, dried blood spots, etc) in most hospitals, laboratories, and screening facilities, to make this determination. If the blood sample is positive, an aliquot of the sample is further tested, using different approved algorithms for confirmation. The presence of antibody in the confirmatory test is considered a positive confirmation and indication that the blood sample donor is infected.

A need exists to develop improved methods and kits that enable efficient diagnosis or detection of antibodies against viral infections.

### SUMMARY OF THE INVENTION

In one embodiment, the invention provides a formulation comprising a medium for incubating a whole blood sample in a culture, wherein said medium comprises (a) one or more activators of (i) virus-primed lymphocytes, (ii) memory cells specific for said virus, (iii) virus-specific antibody production, or (iv) a combination thereof, and (b) a non-carbon dioxide-dependent buffering agent.

In another embodiment, the present invention provides a container apparatus comprising a medium for incubating a whole blood sample in a culture, wherein said medium comprises (a) one or more activators of (i) virus-primed lymphocytes, (ii) memory cells specific for said virus, (iii) virus-specific antibody production, or (iv) a combination thereof and (b) a non-carbon dioxide-dependent buffering agent.

In one embodiment, the invention provides a method for detecting virus-specific antibodies in a whole blood sample from a subject in the absence of CO₂ enrichment, the method comprising: (a) incubating a whole blood sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), thereby detecting the presence of viral-specific antibodies following a culture step in the absence of CO₂ enrichment.

In other embodiments, the method further comprises the steps of: (i) collecting a first blood sample prior to the incubation step and storing said first blood sample; (ii) collecting a second blood sample after said incubation step; (iii) measuring the level of antibodies in said first and said second blood samples concurrently; and (iv) comparing the measurements of the levels of antibodies between said first and second blood samples.

In another embodiment, the invention provides a method for diagnosing a viral infection in a subject, the method comprising: (a) collecting a whole blood sample from said subject; (b) incubating said blood sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (c) exposing the resultant culture of step b) to a viral antigen, thereby allowing an antigen-antibody immune complex to form; and (d) detecting the antigen-antibody immune complex of step c), wherein the detection of said antigen-antibody immune complex indicates said subject has said viral infection.

In another embodiment, the invention provides a method for increasing low virus-specific antibody levels in a whole blood sample from a subject to a detectable level comprising: (a) collecting said whole blood sample from said subject; and (b) incubating said sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; thereby increasing low virus-specific antibody levels to a detectable level in said sample.

In another embodiment, the invention provides a kit for the detection of virus-specific antibodies, the kit comprising: a container for retaining and culturing a whole blood sample, wherein said container comprises a mechanism for incubating said sample without any CO₂ enrichment, and wherein said container further comprises a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

The present invention comprises an improved assay for detecting antibodies in tissue samples of individuals who test seronegative by conventional assay techniques, thus aiding in the diagnosis of possible virus infections. More particularly, the present invention relates to an improved assay methods and kits that enable efficient detection of antibodies against a viral infection.

In one embodiment, the invention provides a formulation comprising a medium for incubating a tissue sample in a culture, wherein said medium comprises (a) one or more activators of (i) virus-primed lymphocytes, (ii) memory cells specific for said virus, (iii) virus-specific antibody production, or (iv) a combination thereof, and (b) a non-carbon dioxide-dependent buffering agent.

In another embodiment, the present invention provides a container apparatus comprising a medium for incubating a tissue sample in a culture, wherein said medium comprises (a) one or more activators of (i) virus-primed lymphocytes, (ii) memory cells specific for said virus, (iii) virus-specific antibody production, or (iv) a combination thereof and (b) a non-carbon dioxide-dependent buffering agent.

In one embodiment, the invention provides a method for detecting virus-specific antibodies in a sample from a subject in the absence of CO₂ enrichment, the method comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), thereby detecting the presence of viral-specific antibodies in the absence of CO₂ enrichment. In one embodiment, only the culture step is performed in the absence of CO₂ enrichment.

In some embodiments, the method further comprises the steps of: (i) collecting a first assay sample prior to the incubation; (ii) measuring the level of antibodies in said first assay sample; (iii) collecting a second assay sample after the incubation; (iv) measuring the level of antibodies in said second assay sample; and (v) comparing the measurements of the levels of antibodies between said first and second assay samples. In one embodiment, the assay sample is a tissue sample. In one embodiment, the assay sample is a blood sample. In one embodiment, the assay sample is a whole blood sample.

In one embodiment, said antigen is added to said culture to shorten the incubation time, to provide diagnosis in situ, or a combination thereof. In another embodiment, the antigen-antibody immune complex is detected on a solid phase support or carrier, which in one embodiment, is a nitrocellulose strip, a set of labeled or colored beads, or any other carrier. In one embodiment, the carrier may comprise beads with different densities, sizes, labels, colors, fluorescence, as is known in the art.

In another embodiment, the invention provides a method for diagnosing a viral infection in a subject, the method comprising: (a) collecting a tissue sample from said subject; (b) incubating said sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (c) exposing the resultant culture of step b) to a viral antigen, thereby allowing an antigen-antibody immune complex to form; and (d) detecting the antigen-antibody immune complex of step c), wherein the detection of said antigen-antibody immune complex indicates said subject has said viral infection.

In another embodiment, the invention provides a method for increasing low virus-specific antibody levels in a tissue sample from a subject to a detectable level comprising: (a) collecting said tissue sample from said subject; and (b) incubating said sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; thereby increasing low virus-specific antibody levels to a detectable level in said sample.

In another embodiment, the invention provides a method for detecting a viral infection in a subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), thereby detecting the presence of viral-specific antibodies in the absence of CO₂ enrichment; thereby detecting a viral infection in said subject.

In another embodiment, the invention provides a method for detecting an early or recent viral infection in a subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), thereby detecting an early or recent viral infection in said subject.

In another embodiment, the invention provides a method of inducing antibody production or expression from virus-primed B cells in a tissue sample from a subject in the absence of CO₂ enrichment comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; and, optionally, (b) exposing the resultant culture of step a) to a viral antigen, thereby allowing an antigen-antibody immune complex to form; and (c) detecting the antigen-antibody immune complex of step b), thereby inducing antibody production or expression from virus-primed B cells in a tissue sample from said subject in the absence of CO₂ enrichment.

In another embodiment, the present invention provides a method of stimulating antibody production from virus-primed B cells in a tissue sample from a subject comprising the steps described hereinabove. In one embodiment, the primed B cells are activated. In another embodiment, the primed B cells are suppressed.

In another embodiment, the invention provides a method for detecting a viral infection in a seronegative subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), wherein if the antigen-antibody immune complex is detected, said seronegative subject has a viral infection, thereby detecting a viral infection in said seronegative subject.

In another embodiment, the invention provides a method for detecting a very recent viral infection in a subject comprising: (a) exposing a tissue sample from a subject to a viral antigen of interest, thereby allowing an antigen-antibody immune complex to form; (b) detecting the antigen-antibody immune complex of step b), (c) incubating a second tissue sample from said subject in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (d) exposing the resultant culture of step c) to a viral antigen of interest, thereby allowing an antigen-antibody immune complex to form; and (e) detecting the antigen-antibody immune complex of step d), wherein if the antigen-antibody immune complex is detected in step (e) and not in step (b), said subject has a very recent viral infection, thereby detecting a very recent viral infection in said subject.

In another embodiment, the invention provides a method for detecting a viral infection in a subject by raising virus-specific antibody levels comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; (b) exposing the resultant culture of step a) to a viral antigen, thereby allowing an antigen-antibody immune complex to form; and (c) detecting the antigen-antibody immune complex of step b), wherein if the antigen-antibody immune complex is detected, said subject has a viral infection, thereby detecting a viral infection in said subject by raising virus-specific antibody levels.

In another embodiment, the invention provides a method for shortening the window period of a viral infection in a subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; and, optionally, (b) exposing the resultant culture of step a) to a viral antigen, thereby allowing an antigen-antibody immune complex to form; and (c) detecting the antigen-antibody immune complex of step b), wherein if the antigen-antibody immune complex is detected, said subject has a viral infection, thereby shortening the window period of a viral infection in said subject. In one embodiment, the subject is seronegative.

In another embodiment, the invention provides a method for resolving the window period of a viral infection in a subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; and, optionally, (b) exposing the resultant culture of step a) to a viral antigen, thereby allowing an antigen-antibody immune complex to form; and (c) detecting the antigen-antibody immune complex of step b), wherein if the antigen-antibody immune complex is detected, said subject has a viral infection, thereby resolving the window period of a viral infection in said subject.

In another embodiment, the invention provides a method for closing the window period of a viral infection in a subject comprising: (a) incubating a tissue sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent; and, optionally, (b) exposing the resultant culture of step a) to a viral antigen, thereby allowing an antigen-antibody immune complex to form; and (c) detecting the antigen-antibody immune complex of step b), wherein if the antigen-antibody immune complex is detected, said subject has a viral infection, thereby closing the window period of a viral infection in said subject.

In one embodiment, the window period is shortened by 95%. In another embodiment, the window period is shortened by 90%. In another embodiment, the window period is shortened by 85%. In another embodiment, the window period is shortened by 80%. In another embodiment, the window period is shortened by 75%. In another embodiment, the window period is shortened by 50%. In another embodiment, the window period is shortened by 25%. In one embodiment, shortening of the window period for an infection to a week, or in another embodiment, to less than a week, is effectively closing the window period.

In another embodiment, the invention provides a method of differentiating between a cleared and chronic viral infection in a subject comprising: (a) obtaining a tissue sample from said subject; (b) determining the anti-viral antibody level in a first aliquot of said sample, wherein a detectable anti-viral antibody level indicates that a subject is seropositive; (c) stimulating a second aliquot of said sample to produce anti-viral antibodies in vitro by incubating said second aliquot in a culture in the presence of a medium comprising one or more activators of (i) lymphocytes primed by said virus, (ii) memory cells specific for said virus, (iii) antibody production against said virus, or (iv) a combination thereof, and a non-carbon dioxide-dependent buffering agent; (d) determining the anti-viral antibody level in said second aliquot of said sample; (e) dividing a value representing the stimulated anti-viral antibody level obtained in step (d) by a value representing the anti-viral antibody level obtained in step (b) for each subject; (f) determining if the quotient obtained in step (d) is above a pre-determined threshold value for each subject, wherein a value below said threshold value indicates that the subject has cleared the viral infection and a value close to said threshold value indicates that the subject has a chronic viral infection, thereby differentiating between a cleared and chronic viral infection in said subject.

In another embodiment, the invention provides a kit for elevating virus-specific antibodies in the absence of CO₂ enrichment to allow detection, the kit comprising: a container for retaining and culturing a tissue sample, wherein said container comprises a mechanism for incubating said sample without any CO₂ enrichment, and wherein said container further comprises a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent.

In another embodiment, the invention provides a kit for the detection of virus-specific antibodies in the absence of CO₂ enrichment, the kit comprising: (a) a container for retaining and culturing a tissue sample, wherein said container comprises a mechanism for incubating said sample without any CO₂ enrichment, and wherein said container further comprises a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent and (b) an assay for the detection of viral-specific antibodies.

In one example, the invention includes a container for collecting, retaining, treating, or culturing blood, or a combination thereof. The container can optionally contain a culture medium. The preferred container is a test tube. Other containers such as a vacuum-tube, a bottle, a well (as part of a multi well plate or as a single well/plate) or a flask, containing an effective concentration of a medium can also be used. The container can be plastic, glass, silicon, synthetic membranes, or metal material (with or without special treatment or coating of the inner or outer surface), or any other material that is compatible with culturing blood. In one embodiment, a coating of the inner surface supports the growth of lymphocytes in culture, which in one embodiment, is called a “tissue culture treated” surface. In another embodiment, a coating of the inner surface is a coating of relevant antigens, which, in one embodiment, may be used to bind to the antibody being assayed in the present invention.

It is to be understood that the present invention also includes blood containing means other then a blood collection tube including, but not limited to, microtiter plates containing wells in which the blood can be incubated, tissue culture flasks, glass flasks such as an erlenmeyer flask, and any other container in which the blood can be cultured.

In one embodiment, the container is a vacuum-tube, which in one embodiment, is a sterile glass or plastic tube with a closure that is evacuated to create a vacuum inside the tube facilitating the draw of a predetermined volume of liquid into the tube.

A container apparatus may include a mechanism for incubating a tissue sample in a culture, in the absence of CO₂ enrichment. The container can be made of a polymer non-toxic to cells in said culture, for example, but are not limited to a polystyrene polymer and a polypropylene polymer. In one embodiment, the container is capable of keeping liquid and vacuum inside said culture container stable. In one embodiment, “capable” describes the ability of the container to maintain a vacuum or to prevent the exit of liquids from the container, when the container is used in a certain way that is specified in the instructions, for e.g. when the top of the container is in a particular position. In another embodiment, the container stably maintains liquid and vacuum inside said culture container.

In another embodiment, the container has a cap, stopper, or lid that has a plurality of positioning mechanisms. In one example, the cap, stopper, or lid has a first and second positioning mechanisms, wherein the first positioning mechanism is capable of being fully closed that facilitates keeping the vacuum inside the container and the second positioning mechanism is capable of providing ventilation and sterile environment, inside the container.

In accordance with the present invention, a blood sample is drawn into a container. The blood sample to be tested is cultured in vitro in the presence of the medium discussed herein. After incubation, an aliquot is taken from the top of the fluid and is then assayed for the presence of desired antibodies using standard Rapid ELISA, Western Blot analysis, a lateral flow, or an immunofluorescence assay, and/or any other antibody detection system, which in one embodiment is a Chemiluminescence, luminescence, or chip system. If the sample is to be assayed at a later date, the supernatant fluid may be collected, and stored at −4° C., −20° C., or −80° C. (i.e., refrigerated or frozen). Results may be confirmed or supported by any other method for the detection of viral specific antibodies, antigens, or genomic sequences.

In one embodiment, the present invention provides compositions comprising and methods using one or more activators of (i) lymphocytes primed by said virus, (ii) memory cells specific for said virus, (iii) antibody production against said virus, or (iv) a combination thereof. In one embodiment, the activator is a lymphocyte primed by a specific virus. In one embodiment, the virus-primed lymphocytes are activated. In another embodiment, the virus-primed lymphocytes are suppressed. In one embodiment, the activator is a lymphocyte activated by the virus.

In some embodiments, the method further comprises the steps of: (i) collecting a first assay sample prior to the incubation; (ii) measuring the level of antibodies in said first assay sample; (iii) collecting a second assay sample after the incubation; (iv) measuring the level of antibodies in said second assay sample; and (v) comparing the measurements of the levels of antibodies between said first and second assay samples.

In other embodiments, the method further comprises the steps of: (i) collecting a first assay sample prior to the incubation step and storing said first assay sample; (ii) collecting a second assay sample after said incubation step; (iii) measuring the level of antibodies in said first and said second assay sample concurrently; and (iv) comparing the measurements of the levels of antibodies between said first and second assay samples.

In one embodiment, “concurrently” refers to running the first assay sample and second assay sample in the same antibody detecting assay on the same day, which, in one embodiment, provides more direct comparative data and provides less assay variation than assays run on separate days. In one embodiment, the first assay sample is stored until after the incubation and collection of the second sample. In one embodiment, the two samples (i.e. before and after incubating with the activator described herein) are taken from the same tube. In one embodiment, the first sample is from time 0days and the second sample is from time Xdays, wherein X is the number of days that the sample is incubated with the activator or activators as described herein.

In one embodiment, the incubation of the blood in the medium comprising activator and a non-carbon dioxide-dependent buffering agent is one day. In another embodiment 2 days. In another embodiment, the incubation is 3 days. In another embodiment, the incubation is 4 days. In another embodiment, the incubation is 5 days. In another embodiment, the incubation is 6 days. In another embodiment, the incubation is 7 days. In another embodiment, the incubation is 3-5 days. In another embodiment, the incubation is 2-7 days.

In one embodiment, the collected tissue sample is incubated without any CO₂ enrichment, in a culture in the presence of a medium. In one example, the incubation is performed in the absence of a CO₂ incubator. In some embodiments, one or more components of the medium are not dependent on CO₂ for buffering. In a particular embodiment, the medium comprises a buffering agent.

Culture medium may refer to any medium that can be used to practice the present invention, without the need for CO₂-enriched environment. In one embodiment, the medium is supplemented with appropriate antibiotics and glutamine.

In one embodiment, the medium comprises a non-carbon dioxide-dependent buffering agent. In one embodiment, the medium comprises a carbon dioxide-independent buffering agent, which in one embodiment, is commercially available, in one embodiment, from Sigma, Gibco, or Invitrogen. In one embodiment, such a medium is buffered by its complement of salts, free base amino acids and galactose substituted for glucose to help maintain physiological pH. In one embodiment, examples of buffering agents include, but are not limited to, HEPES ((4-(2-hydroxyethyl)-1-piperazineethanesulfonic acid) and a phosphate-based buffering agent. In one embodiment, the phosphate-based buffering agent is L15 Medium (Leibovitz).

In one embodiment, the medium comprising a non-carbon dioxide-dependent buffering agent allows incubation of the sample from the subject without the need for a CO₂ incubator. Until now, the incubation step with activator required a CO₂ incubator with 5% CO₂, and the buffering of the media comprised carbonates. The CO₂ incubator requires a CO₂ tank, which needs to be replenished on a regular basis, and might be considered a hazard due to the pressurized gas. It also requires constant accurate monitoring and adjusting, systems which many laboratories do not have. A major advantage of the new formulation and container as described herein is that the buffer has other (or additional) components, so that it maintains an environment which supports complex human tissue culture processes (such as proliferation and differentiation, and high levels of protein secretion), without the enriched 5% CO₂ environment.

In one embodiment, an “activator” for use in the compositions and methods of the present invention is a substance that induces the activation of primed B-cells and/or primed T-cells. In another embodiment an activator induces the activation of memory cells. In another embodiment, an activator induces the activation virus-specific lymphocytes. In another embodiment, an activator induces the activation virus-primed lymphocytes. In another embodiment, an activator induces virus-specific antibody production. In another embodiment, an activator induces virus-specific antibody expression. In one embodiment, virus-specific antibody expression describes expression on the cell membrane. In another embodiment, virus-specific antibody expression describes expression on the cell surface. In another embodiment, an activator induces virus-specific antibody secretion. In another embodiment, an activator induces virus-specific antibody production and expression. In another embodiment, an activator induces virus-specific antibody production, expression, and secretion. In another embodiment, an activator induces virus-specific antibody expression and secretion. In another embodiment, an activator does a combination of the above.

In one embodiment, the substance is a protein or a mixture of proteins, while in another embodiment, the substance is a peptide, a lectin, a nucleic acid molecule, a glycoprotein, a carbohydrate molecule, or a combination thereof. In another embodiment, the substance is a mixture of peptides, nucleic acid molecules, glycoproteins, carbohydrate molecules, or a combination thereof. In one embodiment, “activation” of cells comprises inducing proliferation of cells, differentiation of cells, enhancement of cellular activity, antibody production and antibody secretion, secretion of various lymphokines and/or cytokines, or a combination thereof.

In one embodiment, the activator is a mitogen. In one embodiment, a “mitogen” is a chemical substance, or a mixture of substances. In one embodiment, the mitogen is a biochemical substance, or a mixture of biochemical substances. In one embodiment, the mitogen is a protein that encourages a cell to commence cell division, triggering mitosis. In one embodiment, a mitogen triggers signal transduction pathways in which mitogen-activated protein kinase is involved, leading to mitosis. In one embodiment, mitogens of the present invention are used to induce mitosis, activation, differentiation, or a combination thereof, in memory B cells or virus-primed B cells and/or virus-primed T-cells. In one embodiment, mitogens of the present invention are used to induce the formation of plasma cells from primed differentiating B cells and/or memory B cells. In another embodiment, mitogens of the present invention are used to induce the formation of plasma cells from primed, yet “silenced” or “tolerized”, or suppressed, B-cells and/or memory B cells.

In one embodiment, the mitogen of the compositions and methods of the present invention induces the activation of memory cells and/or in-vivo primed B cells specific for the virus of interest. In another embodiment, the mitogen of the present invention induces the expression of viral-specific antibodies. In another embodiment, the mitogen of the present invention induces the transfer from memory cells and/or in-vivo primed B cells to plasma cells.

In one embodiment, viral antigens are used in conjunction with mitogens to induce activation of memory B cells and/or in-vivo primed B cells. Thus, in one embodiment, the compositions of the present invention, including those for use in the methods of the present invention, additionally comprise an antigen that is specific to the virus of interest which, in one embodiment, aids or enhances the transfer from memory cells, and/or in-vivo primed B cells to plasma cells. Similarly, the methods of the present invention may comprise incubating a blood sample in a medium comprising a mitogen and a viral antigen.

In one embodiment, an antigen is a compound, composition, or substance that can stimulate the production of antibodies or a T cell response in a subject, including compositions that are injected or absorbed into the subject. An antigen reacts with the products of specific humoral or cellular immunity, including those induced by heterologous immunogens. The term “antigen” includes all related antigenic epitopes. “Epitope” or “antigenic determinant” refers to a site on an antigen to which B and/or T cells respond. In one embodiment, T cells respond to the epitope, when the epitope is presented in conjunction with an MHC molecule, or a stand alone, or bound peptide. Epitopes can be formed both from contiguous amino acids or noncontiguous amino acids juxtaposed by tertiary folding of a protein. An epitope typically includes at least 3, and more usually, at least 5, about 9, or about 8-10 amino acids in a unique spatial conformation.

In one embodiment, a peptide is 6-200 amino acids long. In another embodiment, a peptide is 2-50 amino acids. In another embodiment, a peptide is 15-50 amino acids. In another embodiment, a peptide is 1-100 amino acids. In another embodiment, a peptide is about 3-30 amino acids. In another embodiment, a peptide is 5-20 amino acids.

In one embodiment, said antigen is added to said culture to shorten the incubation time and/or to provide diagnosis in situ. In another embodiment, the antigen-antibody immune complex is detected on a solid phase support, carrier, or solid base, which in one embodiment, is a nitrocellulose strip, a set of labeled or colored beads, or any other carrier. In one embodiment, the carrier may comprise beads with different densities, sizes, labels, colors, fluorescence, as is known in the art.

In one embodiment, the methods of the present invention identify a viral infection. According to this aspect and in one embodiment, the antigen used in the methods and kits of the present invention is a viral surface antigen or a viral core antigen.

In one embodiment, a pathogen- or virus-specific antigen is used in the compositions and methods of the present invention. In one embodiment, a pathogen-specific antigen is an immunogenic peptide, which in one embodiment, is a peptide which comprises an allele-specific motif or other sequence such that the peptide will bind an MHC molecule and induce a cytotoxic T lymphocyte (“CTL”) response, or a B cell response (e.g. antibody production or memory B cell proliferation) specific to the antigen from which the immunogenic peptide is derived.

In one embodiment, immunogenic peptides are identified using sequence motifs or other methods, such as neural net or polynomial determinations, known in the art. Typically, algorithms are used to determine the “binding threshold” of peptides to select those with scores that give them a high probability of binding at a certain affinity and will be immunogenic. The algorithms are based either on the effects on MHC binding of a particular amino acid at a particular position, the effects on antibody binding of a particular amino acid at a particular position, or the effects on binding of a particular substitution in a motif-containing peptide. Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Immunogenic peptides can also be identified by measuring their binding to a specific MHC protein (e.g. HLA-A02.01) and by their ability to stimulate CD4 and/or CD8 when presented in the context of the MHC protein.

In one related aspect, the mitogen used in the invention provided herein can be either T-cell dependent or T-cell independent. In one embodiment, the mitogen used in the compositions and methods of the present invention acts on T-cells, B-cells, or both T cells and B cells. In one related aspect, the mitogen used to induce activation of memory B cells and/or in-vivo primed B cells and the expression of virus-specific antibodies is pokeweed mitogen, which in one embodiment, stimulates both B- and T-cells. Other mitogens can be used in practicing the present invention and include, but are not limited to, lectins, such as, concanavalin A, which in one embodiment acts on T cells; bacterial endotoxins, which in one embodiment, is lipopolysaccharide (LPS), which in one embodiment, acts on B cells. In another embodiment, the mitogen is phytohaemagglutinin (PHA), which in one embodiment, acts on T cells. In another embodiment, the mitogen is leukoagglutinin (PHA-L), while in another embodiment, the mitogen is Pisum sativum agglutinin (PSA).

In another embodiment, the activator used in the composition and methods of the present invention is a cytokine, or a mixture of cytokines, which in one embodiment is a signaling molecule secreted by specific cells of the immune system and/or dendritic or glial cells. In one embodiment, said cytokine is an interleukin or interferon. In one embodiment, the cytokine is a lymphokine. In one embodiment, said lymphokine is Interleukin 1, Interleukin 2, Interleukin 3, Interleukin 4, Interleukin 5, Interleukin 6, Interleukin 10, Interleukin 12, Granulocyte-macrophage colony-stimulating factor, Interferon-gamma, or a combination thereof.

In one embodiment, the cytokine is a mediator of adaptive immunity. In another embodiment, the cytokine is a mediator of natural immunity. In another embodiment, the cytokine is tumor necrosis factor (TNF)-α, a type I interferon (IFN) (which, in one embodiment, is IFN-α or IFN-β), or a chemokine. In another embodiment, the cytokine is transforming growth factor (TGF)-β. In another embodiment, the cytokine is a stimulator of hematopoesis, which in one embodiment, is Granulocyte-Macrophage Colony Stimulating Factor (GM-CSF), Macrophage colony-stimulating factor (M-CSF), or Granulocyte colony-stimulating facto (G-CSF). In another embodiment, the cytokine is Interleukin-17.

In another embodiment, the activator used in the compositions and methods of the present invention is a bacterially derived lipid A, a viral-derived peptide, a virus, a biological agent, an anti-immunoglobulin reagent, an antibody against a B-lymphocyte and/or T-lymphocyte cellular domain, or a combination thereof.

In one embodiment, lipid A is a lipid component of an endotoxin held responsible for toxicity of Gram-negative bacteria and a very potent stimulant of the immune system, in one embodiment, activating monocytes or macrophages. In one embodiment, the lipid A has 6 acyl chains. In one embodiment, lipid A is from group of Gram-negative bacteria, including Escherichia coli (E. coli), Salmonella, Shigella, other Enterobacteriaceae, Pseudomonas, Moraxella, Helicobacter, Stenotrophomonas, Bdellovibrio, acetic acid bacteria, Legionella, cyanobacteria, spirochaetes, green sulfur or green non-sulfur bacteria.

In one embodiment, the activator used in the compositions and methods of the present invention is a lectin. In one embodiment, the lectin is a Mannose binding lectin, which in one embodiment, is Concanavalin A, Lentil lectin, or Snowdrop lectin. In another embodiment, the lectin is a Galactose/N-acetylgalactosamine binding lectin, which in one embodiment, is Ricin, Ricinus communis Agglutinin, RCA120; Peanut agglutinin; Jacalin; or Hairy vetch lectin. In another embodiment, the lectin is a N-acetylglucosamine binding lectin, which in one embodiment, is Wheat Germ agglutinin. In another embodiment, the lectin is a N-acetylneuraminic acid binding lectin, which in one embodiment, is Elderberry lectin, Maackia amurensis leukoagglutinin, or Maackia amurensis hemoagglutinin. In another embodiment, the lectin is a Fucose binding lectin, which in one embodiment is Ulex europaeus agglutinin and, in another embodiment, Aleuria aurantia lectin.

In another embodiment, a commercially available container comprising one or more activators as described herein is used as the activator in the compositions and methods of the present invention.

The optimal concentration of mitogen or other activator is easily determined without undue experimentation by one of ordinary skill in the art. In one related aspect, the mitogen concentration range used in the methods provided herein is between approximately 1:10 and 1:5000 dilutions of stock concentration. In another aspect, the concentration range is between 1:100 and 1:500 dilutions of stock. In another embodiment, the mitogen is a lectin, a bacterial endotoxin, a virus, lipid A or a lymphokine. In another aspect, the mitogen is a memory cell affecting mitogen, wherein the memory cell is a B-lymphocyte or a T-lymphocyte memory cell. In another aspect, the mitogen is an antigen-primed cell affecting mitogen, wherein the primed cell is a B-lymphocyte or a T-lymphocyte memory cell.

In one embodiment, the compositions of the present invention and the compositions for use in the methods of the present invention comprise a single activator, which in one embodiment is a mitogen, which in one embodiment, is pokeweed mitogen. In another embodiment, the compositions of the present invention and the compositions for use in the methods of the present invention comprise two activators, in another embodiment, three activators, in another embodiment, four activators, and, in another embodiment, five or more activators. In the case where more than one activator is included, each activator may be from a different class, or each activator may be from the same class. For example, the composition comprises a mitogen, a lymphokine, and a B-lymphocyte cellular domain antibody, which in one embodiment is pokeweed mitogen, interleukin-6, and anti-IgD antibody. In another embodiment, the composition comprises two mitogens, which in one embodiment are pokeweed mitogen and lipopolysaccharide. It is to be understood that any combination of activators as described herein are to be considered part of the invention.

In one related aspect, stimulation of memory cells and/or primed cells is achieved by using antibodies against cellular membrane domains. In another embodiment, memory cells and/or primed cells are stimulated by using antibodies against a B-lymphocyte cellular domain, which in one embodiment is a B-lymphocyte membrane domain. In one embodiment, the antibody is anti-IgD. In one embodiment, IgD is membrane-expressed by naïve B cells, initially primed B cells, and memory cells. In one embodiment, plasma cells do not express membrane IgD. In one embodiment, primed B cells that have not fully differentiated to plasma cells can be stimulated or activated by contacting them with anti-IgD.

In one embodiment, an “antibody” is a polypeptide ligand comprising at least a light chain or heavy chain immunoglobulin variable region which specifically recognizes and binds an epitope (e.g., an antigen, such as a tumor or viral antigen or a fragment thereof). This includes intact immunoglobulins and the variants and portions of them well known in the art, such as Fab′ fragments, F(ab)′.sub.2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (e.g., humanized murine antibodies), heteroconjugate antibodies (e.g., bispecific antibodies). See also, Pierce Catalog and Handbook, 1994-1995 (Pierce Chemical Co., Rockford, Ill.); Kuby, J., Immunology, 3.sup.rd Ed., W.H. Freeman & Co., New York, 1997.

In one aspect, stimulation of virus-primed cells or memory cells results in transformation of the virus-primed cell or the memory cell to an antibody-secreting plasma cell, whereby the plasma cell secretes virus-specific antibodies.

In one embodiment, a virus-primed lymphocyte of the present invention is a B-lymphocyte. In another embodiment, a virus-primed lymphocyte of the present invention is a T-lymphocyte. In one embodiment, a virus-activated lymphocyte of the present invention is a B-lymphocyte. In another embodiment, a virus-activated lymphocyte of the present invention is a T-lymphocyte.

In one embodiment, a memory cell of the present invention is a B-lymphocyte. In another embodiment, a memory cell of the present invention is a T-lymphocyte.

In a related aspect, the B lymphocyte of the methods provided herein is a memory B-lymphocytic cell. In another related aspect, the T lymphocyte is a memory T-lymphocytic cell. In yet another related aspect, the activator provided herein activates a memory B-lymphocytic cell. In another embodiment, the activator activates a memory T-lymphocytic cell, and in another embodiment the activator activates both T and B cells.

In one embodiment, the medium described hereinabove further comprises a pathogen-specific antigen, in one embodiment, a virus-specific antigen, thereby allowing an antigen-antibody immune complex to form. In another embodiment, the medium further comprises a means for detecting an antigen-antibody immune complex. In one embodiment, the pathogen is a virus. In another embodiment, the pathogen is a retrovirus. In another embodiment, the pathogen is a bacteria. In one embodiment, the pathogen or infectious agent, is a microbe or microorganism such as a virus, bacterium, prion, or fungus that causes disease in its animal or human host.

In one embodiment, the viral infection of the present invention is a very early viral infection. In one embodiment, the viral infection of the present invention is an early viral infection. In another embodiment, the viral infection of the present invention is a recent viral infection. In another embodiment, the viral infection of the present invention is a cleared viral infection. In another embodiment, the viral infection of the present invention is a chronic viral infection.

In one embodiment, the viral infection of the present invention is a latent viral infection. In another embodiment, the viral infection of the present invention is a chronic viral infection. In another embodiment, the viral infection of the present invention is a breakthrough viral infection. In another embodiment, the viral infection of the present invention is a viral re-infection. In another embodiment, the viral infection of the present invention is a recent viral infection. In another embodiment, the viral infection of the present invention is a very early viral infection, which in one embodiment, is a window period viral infection. In another embodiment, the viral infection of the present invention is any combination of the above infections. In another embodiment, the viral infection of the present invention is both chronic and latent. In one embodiment, the viral infection of the present invention is chronic and/or latent. In one embodiment, “and/or” refers to either component or a combination thereof, such that a chronic and/or latent infection is a chronic infection, a latent infection, or both a chronic and latent infection. In one embodiment, the viral infection of the present invention is a chronic infection and a co-infection or a super-infection. In one embodiment, the co-infection or super-infection is with the same pathogen or virus. In one embodiment, the co-infection or super-infection is with a different strain or type or subtype of the same pathogen or virus. In another embodiment, the co-infection or super-infection is with a different pathogen or virus.

In another embodiment, the viral infection of the present invention is an infection that is transmitted from a mother to a child.

In one embodiment, the initial viral infection results in the priming of naïve B cells and T-cells, and in the generation of plasma cells and memory B lymphocytes specific for a viral antigen. In another embodiment the initial viral infection results in the priming of naïve B cells (and T cells) yet the generation of plasma cells and/or memory lymphocytes specific for a viral antigen is slowed down, or suppressed, or silenced, or tolerized, or any combination thereof, thus keeping the antibody production levels at low or non-existent levels which cannot be detected by the current antibody detection methods; and can be activated to produce antibody-secreting plasma cells that secrete antigen-specific antibodies. In another related aspect, the antibodies are specific for a viral antigen, protein, peptide, or epitope. In another related aspect, the antigen is a peptide, protein, sugar, component of the virus, etc.

In one embodiment, a viral infection related to the methods and kits of the present invention may be a persistent infection. In one embodiment, persistent infections are infections in which the virus is not cleared but remains in specific cells of infected individuals. Persistent infections often involve stages of both silent and productive infection without rapidly killing or even producing excessive damage of the host cells. In one embodiment, the persistent infection is a latent infection, a chronic infection, or a slow infection. In one embodiment, latent infection is characterized by the lack of demonstrable infectious virus between episodes of recurrent disease. In one embodiment, chronic infection is characterized by the continued presence of infectious virus following the primary infection and can include chronic or recurrent disease. In one embodiment, slow infection is characterized by a prolonged incubation period followed by progressive disease. Unlike latent and chronic infections, slow infection may not begin with an acute period of viral multiplication. During persistent infections, the viral genome can be either stably integrated into the cellular DNA or maintained episomally. In one embodiment, the persistent infection is HIV, human T-Cell lymphotropic virus, Epstein-Barr virus, cytomegalovirus, herpesvirus, varicella-zoster virus, measles, papovavirus, xenotropic murine leukemia virus-related virus (XMRV), prion, hepatitis virus, adenovirus, parvovirus or papillomavirus.

In one embodiment, a viral infection related to the methods and kits of the present invention may exhibit viral latency. In one embodiment, the viral latency is proviral latency and in another embodiment, the viral latency is episomal latency. In one embodiment, the virus is a herpesvirus, which in one embodiment, is varicella zoster virus, and, in another embodiment, is herpes simplex virus (HSV), which in one embodiment is HSV-1, and in another embodiment, HSV-2. In another embodiment, the virus is a gammaherpesvirinae, which in one embodiment is associated with episomal latency established in cells of the immune system. In one embodiment, the viral infection is Epstein-Barr Virus, which establishes episomal latency in B-cells. In another embodiment, the viral infection is cytomegalovirus (CMV), which in one embodiment, establishes latency within T lymphocytes, endothelial cells, and macrophages.

In one embodiment, a virus related to the methods and kits of the present invention is xenotropic murine leukemia virus (XMRV). In another embodiment, the virus is hepatitis A virus (HAV). In another embodiment, the virus is hepatitis B virus (HBV). In another embodiment, the virus is hepatitis C virus (HCV). In another embodiment, the virus is hepatitis D virus (HDV). In another embodiment, the virus is hepatitis E virus (HEV). In another embodiment, the virus is Human T-lymphotrophic virus-1 (HTLV-1). In another embodiment, the virus is any combination of the viruses disclosed hereinabove. In another embodiment, the virus is hepatitis B virus (HBV), hepatitis C virus (HCV), or hepatitis E (HEV) virus.

In another embodiment the virus is human immunodeficiency virus (HIV). In one embodiment, the HIV is HIV-1. In another embodiment, the HIV is HIV-2. In another embodiment, the HIV is HIV-0.

In one embodiment, a virus of the present invention is a retrovirus. In one embodiment, the retrovirus is an Alpharetrovirus, which in one embodiment, is an Avian leukosis virus or Rous sarcoma virus. In another embodiment, the retroviral infection is a Betaretrovirus infection, which in one embodiment, is a Mouse mammary tumour virus or a human analogue thereof. In another embodiment, the retroviral infection is a Gammaretrovirus infection, which in one embodiment, is a Murine leukemia virus, Feline leukemia virus or a human analogue thereof. In another embodiment, the retroviral infection is a Deltaretrovirus infection, which in one embodiment, is a Bovine leukemia virus or a Human T-lymphotropic virus. In another embodiment, the retroviral infection is a Epsilonretrovirus infection, which in one embodiment, is a Walleye dermal sarcoma virus. In another embodiment, the retroviral infection is a Lentivirus infection, which in one embodiment, is a Human immunodeficiency virus 1, Simian immunodeficiency virus, or Feline immunodeficiency virus. In another embodiment, the retroviral infection is a Spumavirus infection, which in one embodiment, is a Simian foamy virus.

In another embodiment, the viral infection is a hepatitis A virus (HAV) infection, hepatitis B virus (HBV) infection, hepatitis C virus infection (HCV), hepatitis D virus (HDV) infection, or hepatitis E virus (HEV) infection.

In another embodiment, the pathogenic agent is any pathogenic agent, including a virus or retrovirus, known in the art to undergo a “hidden” or latent phase and/or a chronic phase. In one embodiment, the virus has low immunogenicity. In one embodiment, the infected subject is immune suppressed, which in one embodiment, is a mild immunosuppression.

In one embodiment, the compositions and methods of the present invention are for detecting antibodies in and for diagnosing subjects with a pathogenic infection, which is, in one embodiment, a viral infection. In one embodiment, the viral infection is detected in subjects that have not seroconverted, which in one embodiment, is a subject who does not test positive for anti-pathogen antibodies on a standard assay (as described in U.S. Pat. No. 6,352,826, which is incorporated by reference herein in its entirety). In one embodiment, the activators described herein elicit anti-pathogen antibodies in a blood sample of a subject who is seronegative, which in one embodiment, means that the subject does not have detectable antibodies to an antigen from the virus with which he has been infected. In one embodiment, seronegativity indicates either a lack of infection or an early stage of infection. According to the latter embodiment, the window period is the period of time between infection with a virus and creation of measurable antibody levels against the virus. For some viruses, the window period is a matter of days or weeks, while for others, the window period is a matter of weeks or months. In one embodiment, the window period may vary by individual, and in particular, depending on their immune state.

In another embodiment, the activators described herein elicit anti-pathogen antibodies in a blood sample of a subject who is seronegative after a chronic infection in which antibody levels decrease to below detection using standard assays or in subjects with a latent infection, in which antibody levels decrease to below detection using standard assays. In all of the above cases, the present invention allows detection of antibodies in seronegative subjects using a special media that does not require a carbon dioxide incubator.

In another embodiment, the methods of the present invention are for detecting antibodies in and for diagnosing subjects who are seropositive for a viral infection. In another embodiment, the methods of the present invention are for detecting antibodies in and for diagnosing subjects who have undergone seroconversion.

In one embodiment, the compositions and methods of the present invention are for detecting antibodies in and for diagnosing subjects with a chronic viral infection, a latent viral infection, a viral re-infection, a viral breakthrough infection, or a combination thereof. In another embodiment, the methods of the present invention are for detecting antibodies in and for diagnosing subjects who are seropositive for the viral infection. In another embodiment, the methods of the present invention are for detecting antibodies in and for diagnosing subjects who have undergone a recent seroconversion.

In one embodiment, virus latency (or viral latency) is the ability of a pathogenic virus to lie dormant within a cell, denoted as the lysogenic part of the viral life cycle. A latent viral infection is a type of persistent viral infection. A latent infection is a phase in the life cycle of certain viruses in which after initial infection, virus production ceases. However, the virus genome is not fully eradicated. The result of this is that the virus can reactivate and begin producing large amounts of viral progeny (the lytic part of the viral life cycle) without the host being infected by a new virus. The virus may stay within the host indefinitely. In one embodiment, virus latency is not identical to clinical latency, in which the virus is undergoing an incubation period but is not dormant.

In one embodiment, a chronic infection refers to a persistent and lasting infection. In one embodiment, the infection lasts more than three months. In another embodiment, the infection lasts more than six months. In another embodiment, the infection last more than nine months. In another embodiment, the infection lasts more than one year.

In one embodiment, a re-infection as described herein, is a second or subsequent infection by the same agent as a first infection. In one embodiment, the re-infection is with human immunodeficiency virus (HIV). In another embodiment, the re-infection is with HCV. In another embodiment, the re-infection is with HBV. In one embodiment, the re-infection is with a different strain of the pathogen or virus. In one embodiment, the infectious agent is a retrovirus, and the re-infection is with a mutant strain, or a different types, clades, or sub-sets of the same retrovirus, and in one embodiment, drug-resistant strain of the retrovirus. In one embodiment, an infection is a super-infection.

In one embodiment, a breakthrough infection as described herein, is an infection that occurred in a subject after vaccination of said subject with a vaccine against the infectious agent. In one embodiment, the vaccination was not a live virus vaccine. In one embodiment, the vaccination was a protein subunit vaccine. In one embodiment, the vaccination was a killed virus vaccine. In one embodiment, the vaccination was a viral mutant vaccine. In another embodiment, the vaccination was any type known in the art. In another embodiment, a breakthrough infection is an infection caused by the shedding of live virus from a vaccine. In one embodiment, a breakthrough infection is a full-blown infection. In another embodiment, a breakthrough infection shows no signs of disease or symptoms in a subject. In another embodiment, a breakthrough infection is mild. In one embodiment, a breakthrough infection may cause significant or serious illness in a person with a compromised immune system, such as, in one embodiment, a subject infected with HBV.

In one embodiment, the terms “antibody” and “immunoglobulin” are used interchangeably herein. These terms are well understood by those in the field, and refer to a glycosylated (comprising sugar moieties) protein consisting of one or more polypeptides that specifically binds an antigen. The basic structural unit of an antibody is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The term “antibody” also includes any protein or peptide-containing molecule that comprises at least a portion of an immunoglobulin molecule, such as, but not limited to, one complementarity determining region (CDR) of a heavy chain or light chain constant region, a framework region, or any portion thereof. Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. Full-length immunoglobulin “light chains” (of about 25 kDa or about 214 amino acids) comprise a variable region of about 110 amino acids at the NH₂-terminus and a kappa or lambda constant region at the COOH-terminus Full-length immunoglobulin “heavy chains” (of about 50 kDa or about 446 amino acids) similarly comprise a variable region (of about 116 amino acids) and one of the aforementioned heavy chain constant regions or classes, e.g., gamma (of about 330 amino acids). The subunit structures and three-dimensional configurations of different classes of immunoglobulins are well known.

In one embodiment, methods and compositions of the present invention comprise anti-IgD, anti-IgG, anti-IgA, anti-IgE. In another embodiment, methods and compositions of the present invention comprise one or more anti-CD antibodies, which in one embodiment, is anti-CD19, anti-CD10, anti-CD23, anti-CD25, or anti-CD40 antibodies. Methods of making these antibodies are well known in the art and the antibodies are commercially available.

In one related aspect, the present invention provides methods that enable an increase in the levels of the anti-viral antibodies, thus increasing the signal while reducing the noise. In another embodiment, the method of the present invention increases signal to noise ratio. In another embodiment, the method of the present invention decreases the rate of false positives. In another embodiment, the method of the present invention increases the levels of anti-viral antibodies without significantly increasing the noise levels. In another embodiment, the method of the present invention increases the levels of anti-viral antibodies without affecting the accuracy of the diagnosis (or diagnostic specificity). In another embodiment, the method of the present invention decrease the levels of false positives compared to the current testing kits/assays. In another embodiment, the method of the present invention increases the diagnostic specificity of the current testing kits/assays.

In one embodiment, a complex between a sample antibody and a supplied antigen can be detected through the use of a probe directed against a serum antibody. The reaction mixture can be exposed to conditions sufficient for formation of a probe/antibody complex. The presence, absence or quantity of the probe, probe/antibody complex, or probe/antibody/antigen complex can then be detected. The probe can be directed against human immunoglobulin. For example, probes directed against human immunoglobulin include, but are not limited to, an antibody, an antigen-binding fragment thereof, an aptamer or an avimer. Detection of the presence, absence or quantity of a complex between a probe/antibody complex or probe/antibody/antigen complex can be according to any suitable means known in the art, as discussed herein. A probe for use with a sample antibody and a supplied antigen can include a label. Probe labels and detection thereof are known in the art.

The biological sample collected by the methods provided herein may be brought into contact with, and immobilized onto, a solid phase support or carrier, such as nitrocellulose, polymer “beads”, chips, or other solid support or matrix, which is capable of immobilizing cells, cell particles, membranes, peptides, or soluble proteins. The support may then be washed with suitable buffers, followed by treatment with the detectably labeled anti-human antibody. The solid phase support may then be washed with buffer a second time to remove unbound antibody. The amount of bound label on the solid support may then be detected by conventional means.

In one embodiment, the container comprising the tissue and the activators may further comprise a label in non-soluble form bound on a solid support, which in one embodiment, enables the subject to receive a diagnosis after an appropriate period of incubation. Such incubation periods are dependent on the label and on the time required for the activation step and are known in the art. In another embodiment, the label is in soluble form.

In one related aspect, methods of identifying antibodies produced by the methods provided herein through their binding affinities or specificities are very well known in the art and include methods such as immunoprecipitation, radioimmunoassay (RIA), enzyme-linked immunoabsorbent assay (ELISA), lab-chip, bio-chip, or nano-based chip. Other well-known methods can be used to determine antibody binding affinities and these methods can be readily used, as will be understood by a skilled artisan. Polymerase chain reaction (PCR) technique may also be used to enhance the detection level of the preliminary incubation sample. It is to be understood that other assays known in the art to obtain or detect antibody interactions can also be used and these include but are not limited to immuno (western) blots, immunofluorescence assays, and the like. It should be understood by one of skill in the art that any number of conventional protein assay formats, particularly immunoassay formats, may be designed to utilize the specific antigens and antibodies of this invention for the detection of anti-viral antibodies and for the detection of a viral infection in a subject. This invention is thus not limited by the selection of the particular assay format and encompasses assay formats which are known to those of skill in the art.

As a matter of convenience, a packaged combination of reagents in predetermined amounts with instructions for performing the diagnostic assay, e.g. kits, are also within the scope of the invention. The present invention includes a kit comprising a blood collection container containing effective concentrations of mitogens or other activators therein. In one embodiment, the kit comprises a non-carbon dioxide-dependent buffering agent. In one embodiment, a non-carbon dioxide-dependent buffering agent refers to a carbon dioxide-independent buffering agent.

In one embodiment, the container of the kit of the present invention is for retaining tissue samples, or in another embodiment, holding, processing, storing, maintaining or collecting tissue samples.

In another related aspect, the present invention's methods coupled with the present invention's kits enable the diagnostic sensitivity of the combined assay to be brought to practical levels, making it feasible for both diagnostics and for introduction into the blood banks to increase the blood safety.

Kits are also provided that are useful as a positive control for the diagnostic assays. For isolation and purification of anti-viral antibodies, the kit can contain viral proteins/antigens coupled to beads (e.g., sepharose beads or nanobeads or other nano-structures). Kits can be provided which contain the antibodies for detection and quantitation of anti-viral antibodies in vitro, e.g. in an ELISA, peptide microarray, bio-chip, or a Western blot. As with the article of manufacture, the kit comprises a container and a label or package insert on or associated with the container. The container holds a composition comprising at least one antigen recognized by the anti-viral antibodies. Additional containers may be included that contain, e.g., diluents and buffers, control antibodies. The label or package insert may provide a description of the composition as well as instructions for the intended in vitro or diagnostic use.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

In one embodiment, a viral infection can be detected using a diagnostic kit. In one embodiment, the diagnostic kit is one currently available. In one embodiment, a kit of the present invention may be used in conjunction with a diagnostic kit for a particular viral infection. In another embodiment, a kit of the present invention may be used in conjunction with a currently available diagnostic kit for a viral infection.

The method of the present invention includes optionally separating the blood cells from the fluid portion of the blood so that the presence of antibodies, or the presence of antibody-producing cells can be determined. The separation of the blood cells from the fluid portion of the blood can be done by any of several methods well known to those of ordinary skill in the art, including centrifugation or density dependent sedimentation. In one embodiment, the blood cells are not physically separated from the fluid. In another embodiment, peripheral blood mononuclear cells (PBMCs), B-lymphocytes and T-lymphocytes may be separated from the blood prior to culture and assay. Methods of B cell and T cell enrichment are well known in the art and can be carried out by methods that include, but are not limited to, density dependent sedimentation, and/or cell sorting/FACS. After incubation of the tissue with the mitogen, fluid from the top of the blood can easily be extracted and tested for antibody. Optionally, the red blood cells can be lysed either by mild osmotic shock or with a mild detergent. In this way, the white blood cells remain viable. Another method would be to sediment the white blood cells via density, or density gradient.

Generally, the results of a test or assay according to the invention can be presented in any of a variety of formats. The results can be presented in a qualitative fashion. For example, the test report may indicate only whether or not a particular virus-specific antibodies were detected, perhaps also with an indication of the limits of detection. The results may be presented in a semi-quantitative fashion. For example, various ranges may be defined, and the ranges may be assigned a score (e.g., 1+ to 4+) that provides a certain degree of quantitative information. Such a score may reflect various factors, e.g., the number of virus detected, the intensity of the signal (which may indicate the level of expression of virus specific B cells, or T cells), etc. The results may be presented in a quantitative fashion, e.g., as a percentage of cells in which the virus specific antibodies are detected, as a viral specific antibody concentration (as determined via different antibody binding/detection assay), etc. As will be appreciated by one of ordinary skill in the art, the type of output provided by a test will vary depending upon the technical limitations of the test and the biological significance associated with detection.

The method of the present invention includes optionally separating the blood cells from the fluid portion of the blood so that the presence of antibodies can be determined. The separation of the blood cells from the fluid portion of the blood can be done by any of several methods well known to those of ordinary skill in the art, including centrifugation or selective sedimentation. It is to be understood, that the blood cells do not need to be physically separated from the fluid. In one embodiment, PBMCs may be separated from the blood prior to culture and assay. After incubation of the tissue with the mitogen, fluid from the top of the blood can easily be extracted and tested for antibody. Optionally, the red blood cells can be lysed either by mild osmotic shock or with a mild detergent. In this way, the white blood cells remain viable.

In one embodiment of the present invention, whole blood is collected in a blood collection tube containing culture medium and mitogen. The blood samples are then incubated with an approximately 1:50-1:500 final dilution of pokeweed mitogen at a concentration of 0.1-2×10⁶ viable cells per ml for four days at 37 .degree. C. in a 3-10% CO₂ humidified atmosphere. The blood is then centrifuged and the supernatant fluid is collected and assayed within approximately 24 hours for reactive antibodies by ELISA, lateral flow, and/or blot techniques. In the alternative, an aliquot of fluid may be taken directly from the sample. Each sample should be screened for antibody by lateral flow (Rapid test) or ELISA first, samples considered positive may then be subjected to an additional test, e.g. blot analysis.

In some embodiments, the methods of the invention comprises the steps of: (i) collecting a first assay sample prior to the incubation; (ii) measuring the level of antibodies in said first assay sample; (iii) collecting a second assay sample after the incubation; (iv) measuring the level of antibodies in said second assay sample; and (v) comparing the measurements of the levels of antibodies between said first and second assay samples.

In one embodiment, the methods of the present invention additionally comprise the step of exposing a separate aliquot from a sample from the subject to the viral antigen. In one embodiment, if an antigen-antibody immune complex is detected in the stimulated but not the unstimulated aliquot, the subject has a very early viral infection, which in one embodiment, is an infection in the window period, which in one embodiment, is called a window period (WP) infection. In another embodiment, if the level of said antigen-antibody immune complex is higher in step c) than in step d), the subject has a recent viral infection. In another embodiment, if the level of said antigen-antibody immune complex is higher in step d) than in step c), the subject has a cleared viral infection. In another embodiment, if the level of said antigen-antibody immune complex is detectable in step d) but not in step c), the subject has a cleared viral infection.

In one embodiment, a cleared viral infection has been cleared by the subject's immune system. In another embodiment, a virus infection is cleared spontaneously. In another embodiment, the virus infection is cleared following one or more therapeutic courses, or in another embodiment, treatments. In another embodiment, if the levels of the antigen-antibody immune complex are lower in the stimulated versus the unstimulated aliquot, the subject has a resolved viral infection. In another embodiment, if the levels of the antigen-antibody immune complex are lower in the stimulated versus the unstimulated aliquot, the subject has recovered from a viral infection.

In another embodiment, the methods of the present invention additionally comprises the step of exposing a separate aliquot from a sample from the subject to the viral antigen, wherein if the level/amount of the antigen-antibody immune complex in the unstimulated and the stimulated aliquot are equivalent, the subject has a chronic and/or long-term viral infection that has not cleared and that is not very early or recent.

In one embodiment, a method of the present invention requires determining the anti-viral antibody level in an aliquot of a blood sample. In one embodiment, an “aliquot” is a portion of the total amount of a blood sample. In one embodiment, the aliquots used in the methods of the present invention are of equal volume or dilution. In one embodiment, duplicate blood samples are used in the methods of the present invention. In one embodiment, the first and second aliquots of a blood sample are portions of a single blood sample drawn from a single subject at a single time point.

In another embodiment, a single aliquot of the tissue or blood sample may be used to determine both “baseline” antibody levels and stimulated antibody levels, wherein a tissue sample, such as blood, is drawn into a container comprising the activator described herein and the cells are sedimented (by regular G force, or by a short centrifugations at low speed). A small aliquot of the plasma supernatant is removed for later testing of the initial levels of virus. The rest is incubated with the activator for several days. The levels of the antibodies against the virus for the aliquot removed at Time0 is measured on the same assay with an aliquot of blood or tissue removed after the incubation. The two measurements are compared. In one embodiment, the delta (in one embodiment, the difference between the two values) is calculated, in another embodiment, the ratio of signals or levels, is calculated, in another embodiment, the ratio of IgM to IgG of antibodies against the virus is calculated, etc. In one embodiment, the ratio between the stimulated and unstimulated aliquots is called the Stimulation Index or SI.

In one embodiment, the SI may be used to determine the time since a viral infection, since there is a larger difference in stimulated vs unstimulated antibody concentration earlier in the infection, both before and after seroconversion, whereas the difference decreases until it disappears as infection progresses until the infection clears, in which case the antibody levels are reversed, now being higher in the unstimulated aliquot and lower in the stimulated aliquot. This behavior of stimulated and unstimulated virus-infected blood may be used to estimate the time since infection.

In addition, the SI or antibody concentration in stimulated vs unstimulated samples may be used to determine the rate of new incidences of a viral infection in a population. In one embodiment, the present invention provides a method of determining the incidence of “new” viral infections in a population. In one embodiment, “new” viral infections are understood to be “recent” viral infections. In one embodiment, recent infections are determined based on a pre-determined SI value and Mean Recency Duration value, which in turn are based on analysis of an initial population in which the recency of infection is known, as is understood by one skilled in the art.

In one embodiment, “new” viral infections are understood to be “very early”, or window period (WP) viral infections. In one embodiment, very early/WP infections are determined based on a very high (or ∞) SI value and Mean Window-Period Duration value, which in turn are based on analysis of a given population in which the WP of infection is known, as is understood by one of skill in the art.

In one embodiment, the Stimulation Index (SI) value describes the level of anti-virus antibody in a non-activated blood sample versus an activated blood sample. In one embodiment, the SI values will be measured with varying sensitivity or amplitude depending on the detection system used. Thus, the SI values considered as “elevated” in accordance with the present invention will depend upon the precise procedure utilized. The SI values can be tested against samples obtained from individuals known to be recently infected with a virus and compared with similar samples obtained from individuals who have an established viral infection such as, but not limited to, individuals who are known to have been infected for at least 1 year or so. Upon comparison of the results, a suitable SI value can be determined which readily distinguishes a recent infection as defined herein from an established/chronic infection. Assay variation can be controlled by using the value from a standard set of sample pairs. A skilled artisan could readily use standard techniques to determine a suitable SI threshold value when using any of a variety of methods of detecting immunoreactivity to a viral antigen. In one embodiment, the methods of the present invention further comprise the step of determining or estimating a threshold SI value, wherein a value within the range of a threshold indicates that the subject was not recently infected and a value above said threshold indicates that the subject was recently infected. In one embodiment, the threshold SI value is the pre-determined threshold used in the methods of the invention.

In one embodiment, the methods of the present invention comprise calculating the mean number of recently infected subjects divided by the number of seropositive subjects, or the number of total tested, and multiplied by the Mean Recency Duration for said threshold. In one embodiment, the methods of the present invention comprise calculating the mean number of recently infected subjects divided by the product of the number of seropositive subjects, or the number of total tested, and the Mean Recency Duration for said threshold. In another embodiment, (the mean number of recently infected subjects/the number of seropositive subjects)×the Mean Recency Duration for the threshold=a measure of the incidence of new viral infections in said population.

In one embodiment, the “Mean Recency Duration” is a pre-determined time period defined as recent for said threshold. In one embodiment, “recency” is described by the “Mean Recency Duration”, which in one embodiment, is the average time period after infection in which there is an SI greater than the threshold value described hereinabove. For example and in one embodiment, the Mean Recency Duration may be 1 year for a SI threshold value of 1.2, which would mean that subjects with an SI value of 1.2 or higher were likely infected within the last year. In one embodiment, the methods of the present invention further comprise the step of determining or estimating the Mean Recency Duration for a specific SI threshold.

In one embodiment, the present invention provides a method of determining the incidence of “new” viral infections in a population. In one embodiment, “new” viral infections are understood to be “recent” viral infections. As described hereinabove, recent infections are determined based on a pre-determined SI value and Mean Recency Duration value, which in turn are based on analysis of an initial population in which the recency of infection is known, as is understood by one of skilled in the art.

In accordance with the present invention, a blood sample is drawn into a test tube, which in one embodiment, is a vacuum-tube, a bottle, a well (as part of a multi well plate or as a single well or plate) or a flask, containing an effective concentration of a solution of a activators (such as lectins, mitogens, cytokines, lymphokines, and combinations thereof as described hereinabove). The blood sample to be tested is cultured in vitro in the presence of any combination of activators of lymphocytes to achieve the same function.

In one embodiment, the step of determining anti-virus antibody levels comprises performing an antibody assay on each aliquot of said blood samples. In one embodiment, an antibody assay comprises exposing each of said blood samples to a viral antigen thereby allowing an antigen-antibody immune complex to form and detecting said antigen-antibody immune complex. In one embodiment, detection of the antigen-antibody immune complex is semi-quantitative.

In one embodiment, a sample of the present invention is obtained from a bodily fluid, such as fresh whole blood in which a single aliquot is activated and the rest of the sample is not activated, as described herein, or in another embodiment, a sample is a pair of plasma samples, in which one of the plasma pair was from activated and the other plasma pair was from non-activated blood. In one embodiment, the plasma and stimulated-plasma (in one embodiment, plasma treated with an activator comprising pokeweed mitogen as described herein) are stored “properly”, which in one embodiment, is at a temperature of 4° C. (for short term storage of days), or in another embodiment, at a temperature of −20° C. or −80° C. (for long term storage of over a week), as is well known in the art.

In one embodiment, the plasma may be stored for up to 2 days. In another embodiment, the plasma may be stored for up to 7 days. In another embodiment, the plasma may be stored for up to 14 days. In another embodiment, the plasma may be stored for up to 1 month. In another embodiment, the plasma may be stored for up to 6 months. In another embodiment, the plasma may be stored for up to 12 months. In another embodiment, the plasma may be stored for up to 24 months. In another embodiment, the plasma may be stored for up to 3 years. In another embodiment, the plasma may be stored for up to 5 years. In another embodiment, the plasma may be stored for up to 10 years. In another embodiment, the plasma may be stored for up to 20 years.

In one embodiment, treating the subject comprises administering one or more anti-retroviral agents for treating a viral-related disease or related disorder.

In one embodiment, the invention provides a method for enabling early treatment of a viral infection or related disease by detecting virus-specific antibodies in a sample from a subject with a viral infection, the method comprising: (a) incubating a tissue sample from said subject, optionally without any CO₂ enrichment, in a culture in the presence of a medium, said medium comprising an activator of (i) virus-primed lymphocytes, (ii) memory cells specific for said virus, (iii) virus-specific antibody production, or (iv) a combination thereof; (b) exposing the resultant culture of step a) to a viral antigen, thereby allowing an antigen-antibody immune complex to form; and (c) detecting the antigen-antibody immune complex of step b), thereby enabling the early treatment of said viral infection or related disease.

In another embodiment, the present invention provides a kit for the detection of virus-specific antibodies in a subject comprising: a container for retaining a whole blood sample, wherein said container comprises a medium, said medium comprising an activator of (i) lymphocytes activated by said virus, (ii) memory cells specific for said virus, (iii) antibodies against said virus, or (iv) a combination thereof. In another embodiment, the kit may additionally comprise a second container lacking said medium for retaining a whole blood sample. In another embodiment, the kit may additionally comprise an assay for the detection of viral-specific antibodies. In another embodiment, the kit may additionally comprise instructions for use.

In another embodiment, the present invention provides an improved assay for the detection of virus-specific antibodies in a subject comprising: a container for retaining a whole blood sample, wherein said container comprises a medium, said medium comprising an activator of (i) lymphocytes activated by said virus, (ii) memory cells specific for said virus, (iii) antibodies against said virus, or (iv) a combination thereof and an assay for the detection of viral-specific antibodies.

In another embodiment, the present invention provides a kit for the diagnosis of a viral infection in a subject comprising: a container for retaining a whole blood sample, wherein said container comprises a medium, said medium comprising an activator of (i) lymphocytes activated by said virus, (ii) memory cells specific for said virus, (iii) antibodies against said virus, or (iv) a combination thereof. In another embodiment, the kit may additionally comprise a second container lacking said medium for retaining a whole blood sample. In another embodiment, the kit may additionally comprise an assay for the detection of viral-specific antibodies. In another embodiment, the kit may additionally comprise instructions for use.

In another embodiment, the present invention provides an improved assay for the diagnosis of a viral infection in a subject comprising: a container for retaining a whole blood sample, wherein said container comprises a medium, said medium comprising an activator of (i) lymphocytes activated by said virus, (ii) memory cells specific for said virus, (iii) antibodies against said virus, or (iv) a combination thereof and an assay for the detection of viral-specific antibodies.

In one embodiment, “treating” refers to either therapeutic treatment or prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The present invention can be used to determine if “hidden” antibodies are present after or during infection by a microorganism including, but not limited to, yeasts, bacteria, viruses, micoplasma, protozoa, and other classes of microorganisms.

In one embodiment, the present invention provides a tissue sample from a subject for evaluation. In one embodiment, the tissue sample is a blood sample. In another embodiment, the tissue sample is a whole blood sample. In another embodiment, the sample comprises cells in blood or saliva from said subject. In another embodiment, the tissue sample is a cheek or tongue swab. In another embodiment, the tissue sample is a biopsy (e.g. lymph node, liver, etc).

As used herein, the term “whole blood” means blood collected with heparin, EDTA, citrate, or any other substance that prevents coagulation and clotting. The term whole blood as used herein also includes blood collected from an animal or human with heparin, ethylenediaminetetraacetate, citrate, or any other substance that prevents coagulation and clotting. “Whole blood” can also mean blood wherein the red blood cells have been lysed while maintaining the viability of the remaining white blood cells.

The term “sample” includes samples present in an individual as well as samples obtained or derived from the individual.

In one related aspect, the term “about” as refers to plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. In one embodiment, the subject is male. In another embodiment, the subject is female. In one embodiment, the subject is a pregnant or lactating female. In another embodiment the subject is a child, a baby, or a new born infant.

In one embodiment, “and/or” as used herein refers to either component or a combination thereof.

In one embodiment, the methods of the present invention comprise the steps described. In another embodiment, the methods of the present invention consist essentially of the steps described. In another embodiment, the methods of the present invention consist of the steps described. In one embodiment, the compositions of the present invention, comprise the elements described. In another embodiment, the compositions of the present invention consist essentially of the elements described. In another embodiment, the compositions of the present invention consist of the elements described.

Additional objects, advantages, and novel features of the present invention will become apparent to one ordinarily skilled in the art upon examination of the examples below. These examples are presented in order to more fully illustrate the preferred embodiments of the invention. They should in no way be construed, however, as limiting the broad scope of the invention.

### EXAMPLES

### Materials and Methods

### Container

A Vacutainer or a Vacuette (commercial vacuum tubes for drawing blood), or any other vacuum maintaining tube, with the Stimulation-media (SM) in it, can be used to draw volume of 0.2-1 ml blood. Such a test tube can have the following modifications: (a) the container can be of PS (polystyrene) or PP (polypropylene), or any other plastic polymer which can be supportive (or non toxic) of the cell culture—and at the same time keep liquid and vacuum inside—stable; (2) the cap, or stopper, or “top” of the tube can have two positions, i.e., the fully closed position coupled with the vacuum machine that keeps the vacuum and the “loose” position that enables ventilation, while keeps the sterility; and (c) the tube can have a flat bottom and/or a wider diameter that can still fit the conveyer belts of the diagnostic mass testing equipment.

The design of the tube enables incubation without any CO₂ incubator or CO₂ enrichment. A tube can have the buffering based on components, so that it would maintain an environment which supports complex human tissue culture processes (such as proliferation and differentiation, and high levels of protein secretion), without the enriched 3-10% CO₂ environment. Such an environment can be created, for example, by: (a) adding HEPES to the carbonate based buffered culture media (SM); (b) using phosphate-based buffers for the SM media, (e.g. L15 Medium (Leibovitz) where the SM is buffered by its complement of salts, free base amino acids and galactose substituted for glucose to help maintain physiological pH control); (c) using any other cell/tissue culture media which is buffered by a complement of salts which are not carbonate based and are CO₂ dependent.

### Medium

The medium has activators in combination with buffering agents.

### Shortening the Incubation Time and Diagnosis In-Situ

Shortening the incubation time and diagnosis in-situ can be accomplished by adding relevant (e.g., HIV/HCV/retrovirus/virus/pathogen/microorganism specific) antigen(s) to the last part of the culture. The antigen-antibody immune complex can be detected in situ on a solid carrier, such as a nitrocellulose strip, which has bound viral antigens, to the tissue culture step 2-24 hours (or 1-5 days) prior to the end of the culture. This provides the antibodies being produced in the tissue sample an enhanced opportunity to bind to the blotted/bound antigens, thus to be developed as soon as the strip is removed from the culture step, by methods known to one skilled in the art.

### Measuring Antibody Levels in Samples

Two samples (e.g., at baseline (time=0 days) and after incubation in container containing activators for X days (time=X days) drawn from the same tube, before and after incubation, can be used for measurements. The blood is drawn into the container, and the cells are sedimented (by regular G force, or by short centrifugations at low speed). A small aliquot of the plasma supernatant is taken for testing of the initial levels of a microorganism (e.g., pathogen/virus/retrovirus/HIV/HCV). The rest is incubated, as is, (with the top loosened up) for several days. The levels of the antibodies against HIV/HCV/retrovirus/virus/pathogen/microorganism are measured on the same assay. The two measurements are compared, for example, by calculating the Δ, or the ratio of signals or levels, or the ratio of IgM to IgG of antibodies against HIV/HCV/retrovirus/virus/pathogen/microorganism, etc.

### Example 1

### Detection of Virus-Specific Antibodies in Human Immunodeficiency Virus (HIV)-Infected Seropositive Subjects

Blood from subjects known to be infected with HIV is collected in anticoagulant, brought to the laboratory, and, within 24 h at room temperature, 1 ml of blood is transferred into a container comprising activators. The rest of the plasma is tested for HIV antibodies and kept for any additional future comparative testing.

Without any CO₂ incubator or external CO₂ enrichment, the blood is incubated in the container containing activator for 3-5 days, and the treated plasma is collected and tested for HIV antibodies in parallel and using the same kit/assay used to test the non-treated plasma.

### Example 2

### Detection of Viral Specific Antibodies in Hepatitis Virus-Infected Seropositive Subjects

Blood from subjects known to be infected with Hepatitis C virus (or alternatively, Hepatitis A virus, Hepatitis B virus, Hepatitis D virus, or Hepatitis E virus) is collected in anticoagulant, brought to the laboratory, and, within 24 h at room temperature, 1 ml of blood is transferred into a container comprising activators. The rest of the plasma is tested for Hepatitis virus related antibodies and kept for any additional future comparative testing.

Without any CO₂ incubator or external CO₂ enrichment, the blood is incubated in the container containing activator for 3-5 days, and the treated plasma is collected and tested for antibodies using the same kit/assay used to test the non-treated plasma.

Antibody production from samples incubated with activators as described herein and in CO₂-independent media, such as Gibco L-15 Media (Table 1A-B), Sigma L-15 Media (Table 2A), Gibco CO₂-independent medium (Table 3A-B), or HEPES (Table 4A-B) without a carbon dioxide incubator compared to non-activated samples incubated in CO₂ dependent media (e.g. DMEM) in a CO₂ incubator (Tables 1A, 2A, 3A, and 4A) and compared to non-activated samples incubated in CO₂-independent media without a carbon dioxide incubator (Tables 1B, 3B, and 4B). The results demonstrate that incubation of a whole blood sample with activators in several CO₂-independent media without a CO₂ incubator increases IgG and IgM expression compared to non-activated blood from the same donor, whether or not the non-activated blood was incubated with or without a CO₂ incubator. These results demonstrate that the advantages of incubating a sample with the activator may unexpectedly be utilized even under conditions where no CO₂ incubator is available.

TABLE 1A
Antibody production from activated samples incubated in Gibco
L-15 Media without CO₂ compared to non-activated samples incubated in
CO₂ dependent media (e.g. DMEM) in a CO₂ incubator

| | | | anti X IgM | | |
|---|---|---|---|---|---|
| Donor | anti X IgG | | | | Cap |
| No. | % of M | delta | % of M | delta | position |
| 43 | 113.53% | 156.25 | 79.29% | −29.6 | open |
| 44 | 116.36% | 113.34 | 126.81% | 17.4 | open |
| 45 | 114.80% | 110.47 | ND | ND | open |
| 93 | 137.84% | 276 | 120.20% | 40.16 | open |

M = control culture, with CO₂ dependent media, with no activators for antibody production. Activation is measured with respect to the Ab levels in that culture.
Delta = the difference in the levels of anti X IgG or IgM, expressed in Units.
ND = not done

TABLE 1B
Antibody production from activated samples incubated in Gibco
L-15 Media without CO₂ compared to non-activated samples incubated in
CO₂ independent media without CO₂

| | anti X IgG | | anti X IgM | | |
|---|---|---|---|---|---|
| Donor | % of M | | % of M | | Cap |
| No. | w/o | delta | w/o | delta | position |
| 38 | 122.48% | 798.12 | 106.49% | 3 | Close |
| 43 | 105.90% | 73.03 | 119.78% | 18.71 | open |
| 45 | 122.89% | 159.64 | ND | ND | open |
| 93 | 120.65% | 172.06 | 107.25% | 16.16 | open |
| 92 | 106.33% | 31.6 | 92.97% | −6.46 | open |

M w/o = control culture, CO₂ independent media, with no activators for antibody production. Activation is measured with respect to the Ab levels in that culture.
Delta = the difference in the levels of anti X IgG or IgM, expressed in Units.
ND = not done

TABLE 2A
Antibody production from activated samples incubated in Sigma
L-15 Media without CO₂ compared to non-activated samples incubated in
CO₂ dependent media (e.g. DMEM) in a CO₂ incubator

anti X IgM

| | | |
|---|---|---|
| Donor | anti X IgG | Cap |

| No. | % of M | delta | % of M | delta | position |
|---|---|---|---|---|---|
| 82 | 117.70% | 180.93 | 190.0% | 21.4 | Close |
| 82 | 128.32% | 289.49 | 194.2% | 22.4 | open |
| 43 | 111.90% | 137.42 | 129.1% | 41.6 | open |
| 44 | 208.24% | 750.1 | 138.9% | 25.2 | open |

M = control culture, with CO₂ dependent media, with no activators for antibody production. Activation is measured with respect to the Ab levels in that culture.
Delta = the difference in the levels of anti X IgG or IgM, expressed in Units.

TABLE 3A
Antibody production from activated samples incubated
in Gibco CO₂ independent medium without CO₂ compared to
non-activated samples incubated in CO₂ dependent media
(e.g. DMEM) in a CO₂ incubator

| | | | anti X IgM | | |
|---|---|---|---|---|---|
| Donor | anti X IgG | | | | Cap |
| No. | % of M | delta | % of M | delta | position |
| 43 | 114.56% | 168.13 | 99.58% | −0.6 | open |
| 44 | 153.33% | 369.58 | 113.85% | 8.99 | open |
| 92 | 122.03% | 122 | 110.72% | 10.05 | open |

M = control culture, with CO₂ dependent media, with no activators for antibody production. Activation is measured with respect to the Ab levels in that culture.
Delta = the difference in the levels of anti X IgG or IgM, expressed in Units.

TABLE 3B
Antibody production from activated samples incubated
in Gibco CO₂ independent medium without CO₂ incubation
compared to non-activated samples incubated in CO₂ independent
media without CO₂ incubation

| | anti X IgG | | anti X IgM | | |
|---|---|---|---|---|---|
| Donor | % of M | | % of M | | Cap |
| No. | w/o | delta | w/o | delta | position |
| 38 | 109.81% | 288.54 | 157.59% | 26.25 | open |
| 44 | 136.81% | 285.88 | 106.99% | 4.83 | open |
| 92 | 107.12% | 44.92 | 102.82% | 2.85 | open |

M w/o = control culture, CO₂ independent media, with no activators for antibody production. Activation is measured with respect to the Ab levels in that culture.
Delta = the difference in the levels of anti X IgG or IgM, expressed in Units.
ND = not done

TABLE 4A
Antibody production from activated samples incubated in HEPES
Media without CO₂ compared to non-activated samples incubated in CO₂
dependent media (e.g. DMEM) in a CO₂ incubator

| | | anti X IgM | |
|---|---|---|---|
| Donor | anti X IgG | | Cap |

| No. | % of M | delta | % of M | delta | position |
|---|---|---|---|---|---|
| 21 | ND | ND | 174.4% | 91.1 | open |
| 98 | 94.35% | −35.18 | 129.7% | 9.17 | Close |
| 98 | 122.6% | 140.64 | 155.3% | 17.1 | open |
| 82 | ND | ND | 133.3% | 7.91 | Close |
| 82 | 105.8% | 59.61 | 92.5% | −1.8 | open |

M = control culture, with CO₂ dependent media, with no activators for antibody production. Activation is measured with respect to the Ab levels in that culture.
Delta = the difference in the levels of anti X IgG or IgM, expressed in Units.
ND = not done

TABLE 4B
Antibody production from activated samples incubated
in HEPES Media without CO₂ compared to non-activated samples
incubated in CO₂ independent media without CO₂

| | | | anti X IgM | | |
|---|---|---|---|---|---|
| Donor | anti X IgG | | | | Cap |
| No. | % of M | delta | % of M | delta | position |
| 21 | ND | ND | 184.18% | 97.58 | open |
| 98 | 69.02% | −263.7 | 142.84% | 12.005 | Close |
| 98 | 97.95% | −15.98 | 170.60% | 19.826 | open |
| 82 | ND | ND | 125.30% | 6.4 | Close |
| 82 | 115.34% | 143.86 | 148.25% | 7.16 | open |
| 38 | 127.02% | 686.84 | 128.03% | 7.99 | Close |
| 44 | 134.95% | 207.11 | 105.77% | 3.43 | open |
| 36 | 124.60% | 85.41 | 111.84% | 2.67 | open |
| 92 | 124.24% | 109.47 | 137.01% | 28.85 | open |

M w/o = control culture, CO₂ independent media, with no activators for antibody production. Activation is measured with respect to the Ab levels in that culture.
Delta = the difference in the levels of anti X IgG or IgM, expressed in Units.
ND = not done

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A formulation comprising a medium for incubating a whole blood sample in a culture, wherein said medium comprises (a) one or more activators of (i) virus-primed lymphocytes, (ii) memory cells specific for said virus, (iii) virus-specific antibody production, or (iv) a combination thereof, and (b) a non-carbon dioxide-dependent buffering agent.

2. The formulation of claim 1, wherein said buffering agent is 4-(2-hydroxyethyl)-1-piperazineethanesulfonic acid (HEPES).

3. The formulation of claim 1, wherein said buffering agent is a phosphate-based buffer.

4. The formulation of claim 3, wherein said phosphate-based buffer is L15 Medium.

5. The formulation of any one of the preceding claims, wherein said activator is pokeweed mitogen.

6. The formulation of any one of the preceding claims, wherein said activator is a viral-derived peptide, lectin, bacterial endotoxin, a virus, lipid A, a cytokine, a lymphokine, or a combination thereof.

7. A container apparatus comprising a medium for incubating a whole blood sample in a culture, wherein said medium comprises (a) one or more activators of (i) virus-primed lymphocytes, (ii) memory cells specific for said virus, (iii) virus-specific antibody production, or (iv) a combination thereof and (b) a non-carbon dioxide-dependent buffering agent.

8. The container of claim 7, wherein said buffering agent is 4-(2-hydroxyethyl)-1-piperazineethanesulfonic acid (HEPES).

9. The container of claim 7, wherein said buffering agent is a phosphate-based buffer.

10. The container of claim 9, wherein said phosphate-based buffer is L15 Medium.

11. The container of any one of claims 7-10, wherein said activator is pokeweed mitogen.

12. The container of any one of claims 7-10, wherein said activator is a viral-derived peptide, lectin, bacterial endotoxin, a virus, lipid A, a cytokine, or a lymphokine.

13. The container of any one of claims 7-12, wherein said container comprises a polymer that is non-toxic to cells in said culture.

14. The container of claim 13, wherein said polymer is a polystyrene polymer.

15. The container of claim 13, wherein said polymer is a polypropylene polymer.

16. The container of any one of claims 7-15, wherein said container is capable of stably maintaining liquid and/or vacuum pressure inside said container.

17. The container of any one of claims 7-16, wherein said container has a cap that has a plurality of positioning mechanisms.

18. The container of claim 17, wherein said container has a first and second positioning mechanisms, wherein said first positioning mechanism renders the container fully closed, thereby stably maintaining liquid and/or vacuum pressure inside said container and wherein said second positioning mechanism provides ventilation to the contents of said container while preserving a sterile environment inside said container.

19. The container of any one of claims 7-18, wherein said container is made of a plastic, glass, silicon, synthetic membrane, or metal.

20. The container of any one of claims 7-19, wherein said container comprises a treatment and/or coating of the inner surface.

21. The container of any one of claims 7-20, wherein said container is a test tube, a bottle, a well or a flask.

22. The container of any one of claims 7-21, wherein said container is vacuum-sealed.

23. The container of claim 22, wherein said container is a vacutube.

24. A method for detecting virus-specific antibodies in a sample from a subject in the absence of CO₂ enrichment, the method comprising:
a) incubating a whole blood sample from said subject in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent;
b) exposing the resultant culture of step a) to a viral antigen, thereby allowing an antigen-antibody immune complex to form; and
c) detecting the antigen-antibody immune complex of step b);
thereby detecting the presence of viral-specific antibodies following a culture step in the absence of CO2 enrichment.

25. The method of claim 24, wherein said incubating step is performed in the absence of a CO₂ incubator.

26. The method of any one of claims 24-25, wherein said buffering agent is HEPES ((4-(2-hydroxyethyl)-1-piperazineethanesulfonic acid).

27. The method of any one of claims 24-26 wherein said buffering agent is a phosphate-based buffering agent.

28. The method of claim 27, wherein said phosphate-based buffer is L15 Medium.

29. The method of any one of claims 24-28 wherein the culture of step a) results in a supernatant, and the supernatant is exposed to a viral antigen in step b), thereby allowing an antigen-antibody immune complex to form.

30. The method of any one of claims 24-29 wherein said virus is a retrovirus.

31. The method of claim 30, wherein said retrovirus is human immunodeficiency virus (HIV).

32. The method of any one of claims 24-29 wherein said virus is a xenotropic murine leukemia virus (XMRV).

33. The method of any one of claims 24-29 wherein said virus is a hepatitis C virus (HCV).

34. The method of any one of claims 24-29 wherein said virus is a hepatitis B virus (HBV).

35. The method of any one of claims 24-29 wherein said virus is a hepatitis A virus (HAV), hepatitis D virus (HDV), or hepatitis E virus (HEV).

36. The method of any one of claims 24-29 wherein the culture of step a) further comprises an antibody against a B-lymphocyte membrane domain.

37. The method of claim 36, wherein said antibody is anti-IgD.

38. The method of claim 36, wherein said antibody is anti-IgG, anti-IgA or anti-IgE.

39. The method of claim 36, wherein said antibody is anti-CD19, anti-CD10, anti-CD23, anti-CD25, or anti-CD40.

40. The method of any one of claims 24-39, wherein said activator is pokeweed mitogen.

41. The method of any one of claims 24-39, wherein said activator is a viral-derived peptide, lectin, bacterial endotoxin, a virus, lipid A, a cytokine, or a lymphokine.

42. The method of any one of claims 24-41, wherein said virus-primed lymphocytes or memory cells are B-lymphocytes.

43. The method of any one of claims 24-42, wherein said virus-primed lymphocytes or memory cells are T-lymphocytes.

44. The method of any one of claims 24-43, further comprising the step of collecting whole blood sample from said subject, prior to step a).

45. The method of claim 44, wherein said whole blood sample is collected into a container.

46. The method of claim 45, wherein said container comprises said medium.

47. The method of any one of claims 45-46, wherein said container comprises a polymer that is non-toxic to cells in said culture.

48. The method of claim 47, wherein said polymer is a polystyrene polymer.

49. The method of claim 47, wherein said polymer is a polypropylene polymer.

50. The method of any one of claims 45-49, wherein said container is capable of stably maintaining liquid and/or vacuum pressure inside said container.

51. The method of any one of claims 45-50, wherein said container has a cap that has a plurality of positioning mechanisms.

52. The method of claim 51, wherein said container has a first and second positioning mechanisms, wherein said first positioning mechanism renders the container fully closed, thereby stably maintaining liquid and/or vacuum pressure inside said container and wherein said second positioning mechanism provides ventilation to the contents of said container while preserving a sterile environment inside said container.

53. The method of any one of claims 24-52, wherein the method further comprises the steps of: (i) collecting a first blood sample prior to the incubation step; (ii) measuring the level of antibodies in said first blood sample; (iii) collecting a second blood sample after the incubation; (iv) measuring the level of antibodies in said second blood sample; and (v) comparing the measurements of the levels of antibodies between said first and second blood samples.

54. The method of any one of claims 24-52, wherein the method further comprises the steps of: (i) collecting a first blood sample prior to the incubation step and storing said blood assay sample; (ii) collecting a second blood sample after said incubation step; (iii) measuring the level of antibodies in said first and said second blood samples concurrently; and (iv) comparing the measurements of the levels of antibodies between said first and second blood samples.

55. The method of any one of claims 24-54, wherein said antigen is added to said culture to shorten the incubation time and/or to provide diagnosis in situ.

56. The method of any one of claims 24-55, wherein said antigen-antibody immune complex is detected on a solid support.

57. The method of claim 56, wherein said solid support is a nitrocellulose strip.

58. The method of any one of claims 24-57, wherein said subject has a suspected viral infection.

59. A method for diagnosing a viral infection caused by a virus in a subject, the method comprising:
a) collecting a whole blood sample from said subject;
b) incubating said blood sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent;
c) exposing the resultant culture of step b) to an antigen of said virus, thereby allowing an antigen-antibody immune complex to form; and
d) detecting the antigen-antibody immune complex of step c);
wherein the detection of said antigen-antibody immune complex indicates said subject has been infected with said virus.

60. The method of claim 59, wherein said viral infection is a chronic viral infection, a recent viral infection, a very early (WP) viral infection, a latent viral infection, a viral re-infection, a viral breakthrough infection, or a combination thereof.

61. The method of any one of claims 59-60, wherein said incubating step is performed in the absence of a CO₂ incubator.

62. The method of any one of claims 59-61, wherein said buffering agent is HEPES ((4-(2-hydroxyethyl)-1-piperazineethanesulfonic acid).

63. The method of any one of claims 59-61, wherein said buffering agent is a phosphate-based buffering agent.

64. The method of claim 63, wherein said phosphate-based buffer is L15 Medium.

65. The method of any one of claims 59-64, wherein the culture of step b) results in a supernatant, and the supernatant is exposed to a viral antigen in step c), thereby allowing an antigen-antibody immune complex to form.

66. The method of any one of claims 59-65, wherein said virus is a retrovirus.

67. The method of claim 66, wherein said retrovirus is human immunodeficiency virus (HIV).

68. The method of any one of claims 59-65, wherein said virus is a xenotropic murine leukemia virus (XMRV).

69. The method of any one of claims 59-65, wherein said virus is a hepatitis C virus (HCV).

70. The method of any one of claims 59-65, wherein said virus is a hepatitis B virus (HBV).

71. The method of any one of claims 59-65, wherein said virus is a hepatitis A virus (HAV), hepatitis D virus (HDV), or hepatitis E virus (HEV).

72. The method of any one of claims 59-71, wherein the culture of step b) further comprises an antibody against a B-lymphocyte membrane domain.

73. The method of claim 72, wherein said antibody is anti-IgD.

74. The method of claim 72, wherein said antibody is anti-IgG, anti-IgA or anti-IgE.

75. The method of claim 72, wherein said antibody is anti-CD19, anti-CD10, anti-CD23, anti-CD25, or anti-CD40.

76. The method of any one of claims 59-75, wherein said activator is pokeweed mitogen.

77. The method of any one of claims 59-75, wherein said activator is a viral-derived peptide, lectin, bacterial endotoxin, a virus, lipid A, a cytokine, or a lymphokine.

78. The method of any one of claims 59-77, wherein said virus-primed lymphocytes or memory cells are B-lymphocytes.

79. The method of any one of claims 59-78, wherein said virus-primed lymphocytes or memory cells are T-lymphocytes.

80. The method of any one of claims 59-79, wherein said whole blood sample is collected into a container.

81. The method of claim 80, wherein said container comprises said medium.

82. The method of any one of claims 80-81, wherein said container comprises a polymer that is non-toxic to cells in said culture.

83. The method of claim 82, wherein said polymer is a polystyrene polymer.

84. The method of claim 82, wherein said polymer is a polypropylene polymer.

85. The method of any one of claims 80-84, wherein said container is capable of stably maintaining liquid and/or vacuum pressure inside said container.

86. The method of any one of claims 80-85, wherein said container has a cap that has a plurality of positioning mechanisms.

87. The method of claim 86, wherein said container has a first and second positioning mechanisms, wherein said first positioning mechanism renders the container fully closed, thereby stably maintaining liquid and/or vacuum pressure inside said container and wherein said second positioning mechanism provides ventilation to the contents of said container while preserving a sterile environment inside said container.

88. The method of any one of claims 59-87, wherein the method further comprises the steps of: (i) collecting a first blood sample prior to the incubation step; (ii) measuring the level of antibodies in said first blood sample; (iii) collecting a second blood sample after the incubation; (iv) measuring the level of antibodies in said second blood sample; and (v) comparing the measurements of the levels of antibodies between said first and second blood samples.

89. The method of any one of claims 59-87, wherein the method further comprises the steps of: (i) collecting a first blood sample prior to the incubation step and storing said first blood sample; (ii) collecting a second blood sample after said incubation step; (iii) measuring the level of antibodies in said first and said second blood samples concurrently; and (iv) comparing the measurements of the levels of antibodies between said first and second blood samples.

90. The method of any one of claims 59-89, wherein said antigen is added to said culture to shorten the incubation time and/or to provide diagnosis in situ.

91. The method of any one of claims 59-90, wherein said antigen-antibody immune complex is detected on a solid base.

92. The method of claim 91, wherein said solid base is a nitrocellulose strip.

93. A method for increasing low virus-specific antibody levels in a whole blood sample from a subject to a detectable level comprising:
a) collecting said whole blood sample from said subject; and
b) incubating said sample, in the absence of CO₂ enrichment, in a culture in the presence of a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent;
thereby increasing low virus-specific antibody levels to a detectable level in said sample.

94. The method of claim 93, wherein said incubating step is performed in the absence of a CO₂ incubator.

95. The method of any one of claims 93-94, wherein said buffering agent is HEPES ((4-(2-hydroxyethyl)-1-piperazineethanesulfonic acid).

96. The method of any one of claims 93-94, wherein said buffering agent is a phosphate-based buffering agent.

97. The method of claim 96, wherein said phosphate-based buffer is L15 Medium.

98. The method of any one of claims 93-97, wherein said virus is a retrovirus.

99. The method of claim 98, wherein said retrovirus is human immunodeficiency virus (HIV).

100. The method of any one of claims 93-97, wherein said virus is a xenotropic murine leukemia virus (XMRV).

101. The method of any one of claims 93-97, wherein said virus is a hepatitis C virus (HCV).

102. The method of any one of claims 93-97, wherein said virus is a hepatitis B virus (HBV).

103. The method of any one of claims 93-97, wherein said virus is a hepatitis A virus (HAV), hepatitis D virus (HDV), or hepatitis E virus (HEV).

104. The method of any one of claims 93-103, wherein the culture of step b) further comprises an antibody against a B-lymphocyte membrane domain.

105. The method of claim 104, wherein said antibody is anti-IgD.

106. The method of claim 104, wherein said antibody is anti-IgG, anti-IgA or anti-IgE.

107. The method of claim 104, wherein said antibody is anti-CD19, anti-CD10, anti-CD23, anti-CD25, or anti-CD40.

108. The method of any one of claims 93-107, wherein said activator is pokeweed mitogen.

109. The method of any one of claims 93-107, wherein said activator is a viral-derived peptide, lectin, bacterial endotoxin, a virus, lipid A, a cytokine, or a lymphokine.

110. The method of any one of claims 93-109, wherein said virus-primed lymphocytes or memory cells are B-lymphocytes.

111. The method of any one of claims 93-110, wherein said virus-primed lymphocytes or memory cells are T-lymphocytes.

112. The method of any one of claims 93-111, wherein said whole blood sample is collected into a container.

113. The method of claim 112, wherein said container comprises said medium.

114. The method of any one of claims 112-113, wherein said container comprises a polymer that is non-toxic to cells in said culture.

115. The method of claim 114, wherein said polymer is a polystyrene polymer.

116. The method of claim 114, wherein said polymer is a polypropylene polymer.

117. The method of any one of claims 112-116, wherein said container is capable of stably maintaining liquid and/or vacuum pressure inside said container.

118. The method of any one of claims 112-117, wherein said container has a cap that has a plurality of positioning mechanisms.

119. The method of claim 118, wherein said container has a first and second positioning mechanisms, wherein said first positioning mechanism renders the container fully closed, thereby stably maintaining liquid and/or vacuum pressure inside said container and wherein said second positioning mechanism provides ventilation to the contents of said container while preserving a sterile environment inside said container.

120. The method of any one of claims 93-119, wherein said subject has a suspected viral infection.

121. A kit for the detection of virus-specific antibodies, the kit comprising: a container for retaining and culturing a whole blood sample, wherein said container comprises a mechanism for incubating said sample without any CO₂ enrichment, and wherein said container further comprises a medium, said medium comprising one or more activators of (i) lymphocytes primed by said virus, (ii) memory cells specific for said virus, (iii) antibody production against said virus, or (iv) a combination thereof, and a non-carbon dioxide-dependent buffering agent.

122. The kit of claim 121, wherein said kit additionally comprises an assay for the detection of viral-specific antibodies.

123. The kit of claim 122, wherein said assay is an enzyme linked immunosorbent assay, a western blot, a lateral flow, or an immunofluorescence assay.

124. The kit of any one of claims 121-123, wherein said buffering agent is HEPES ((4-(2-hydroxyethyl)-1-piperazineethanesulfonic acid).

125. The kit of any one of claims 121-123, wherein said buffering agent is a phosphate-based buffering agent.

126. The kit of claim 125, wherein said phosphate-based buffer is L15 Medium.

127. The kit of any one of claims 121-126, wherein said virus is a retrovirus.

128. The kit of claim 127, wherein said retrovirus is human immunodeficiency virus (HIV).

129. The kit of any one of claims 121-126, wherein said virus is a xenotropic murine leukemia virus (XMRV).

130. The kit of any one of claims 121-126, wherein said virus is a hepatitis C virus (HCV).

131. The kit of any one of claims 121-126, wherein said virus is a hepatitis B virus (HBV).

132. The kit of any one of claims 121-126, wherein said virus is a hepatitis A virus (HAV), hepatitis D virus (HDV), or hepatitis E virus (HEV).

133. The kit of any one of claims 121-132, wherein the medium further comprises an antibody against a B-lymphocyte membrane domain.

134. The kit of claim 133, wherein said antibody is anti-IgD.

135. The kit of claim 133, wherein said antibody is anti-IgG, anti-IgA or anti-IgE.

136. The kit of claim 133, wherein said antibody is anti-CD19, anti-CD10, anti-CD23, anti-CD25, or anti-CD40.

137. The kit of any one of claims 121-136, wherein said activator is pokeweed mitogen.

138. The kit of any one of claims 121-136, wherein said activator is a viral-derived peptide, lectin, bacterial endotoxin, a virus, lipid A, a cytokine, or a lymphokine.

139. The kit of any one of claims 121-138, wherein said virus-primed lymphocytes or memory cells are B-lymphocytes.

140. The kit of any one of claims 121-139, wherein said virus-primed lymphocytes or memory cells are T-lymphocytes.

141. The kit of any one of claims 121-140, wherein said container comprises a polymer non-toxic to cells in said culture.

142. The kit of claim 141, wherein said polymer is a polystyrene polymer.

143. The kit of claim 141, wherein said polymer is a polypropylene polymer.

144. The kit of any one of claims 121-143, wherein said container is capable of stably maintaining liquid and/or vacuum pressure inside said container.

145. The kit of any one of claims 121-143, wherein said container has a cap that has a plurality of positioning mechanisms.

146. The kit of claim 145, wherein said container has a first and second positioning mechanisms, wherein said first positioning mechanism renders the container fully closed, thereby stably maintaining liquid and/or vacuum pressure inside said container and wherein said second positioning mechanism provides ventilation to the contents of said container while preserving a sterile environment inside said container.

147. The kit of any one of claims 121-146, wherein said container is made of a plastic, glass, silicon, synthetic membrane, or metal.

148. The kit of any one of claims 121-147, wherein said container has a treatment and/or coating of the inner surface.

149. The kit of any one of claims 121-148, wherein said container is a test tube, a bottle, a well or a flask.

150. The kit of any one of claims 121-149, wherein said container is vacuum sealed.

151. The kit of any one of claims 121-150, wherein said container is a vacutube.
